(19) United States
(12) Patent Application Publication
(10) Pub. No.: US 2009/0110143 A1
(43) Pub. Date: Apr. 30, 2009

(54) INSPECTION SYSTEM, INSPECTION METHOD, CT APPARATUS AND DETECTION DEVICE

(76) Inventors: Li ZHANG, Beijing (CN); Zhiqiang CHEN, Beijing (CN); Haifeng HU, Beijing (CN); Yuanjing LI, Beijing (CN); Yinong LIU, Beijing (CN); Shangmin SUN, Beijing (CN); Wenyu ZHANG, Beijing (CN); Yuxiang XING, Beijing (CN)

Correspondence Address: WESTMAN CHAMPLIN & KELLY, P.A., SUITE 1400, 900 SECOND AVENUE SOUTH, MINNEAPOLIS, MN, 55402, US

(21) Appl. No.: 12/254,231

(22) Filed: Oct. 20, 2008

(30) Foreign Application Priority Data
Oct. 30, 2007 (CN) 200710176528.4

(51) Int. Cl. A61B 6/00

(52) U.S. Cl. 378/20

(57) ABSTRACT

An inspection system is disclosed. The system comprises a CT apparatus. The CT apparatus includes a gantry, a radiation source connected with the gantry, a detection device connected with the gantry substantially opposite the radiation source, and a transfer device for transferring an object under inspection. The detection device comprises N rows of detectors arranged at predetermined intervals, where N is an integer greater than 1. With the inspection system according to the present invention, the CT apparatus can perform scanning imaging at a high rate to enable the CT apparatus and an scanning imaging device for obtaining a two-dimensional image of an object under inspection to simultaneously operate, thereby compensating each other's insufficiency.

### BACKGROUND OF THE INVENTION

1. Field of the Invention

The present invention relates to an inspection system, an inspection method, a computed tomography (CT) apparatus and a detection device.

2. Description of the Related Art

Conventionally, a plurality of rows of detectors are used to collect data of a plurality of rows of cross-sections of an object under inspection at one time in order to improve the speed of a CT apparatus, such as the one in patent application WO2005/119297. However, it is not very practical to increase the number of rows of detectors considerably since the cost of the detectors is high.

### SUMMARY OF THE INVENTION

It is an object of the present invention to provide an inspection system, an inspection method, a CT apparatus and a detection device. The detection device is capable of decreasing the number of rows of detectors effectively with an effective detection area of the detection device being increased. Therefore, the cost of the detection device is reduced.

In accordance with an aspect of the present invention, there is provided an inspection system comprising a CT apparatus. The CT apparatus includes a gantry, a radiation source connected with the gantry, a detection device connected with the gantry substantially opposite to the radiation source, and a transfer device for transferring an object under inspection. The detection device comprises N rows of detectors arranged at predetermined intervals, where N is an integer greater than 1.

The predetermined interval may be at least about 5 mm and at most about 80 mm or may be at least about 30 mm and at most about 50 mm.

In accordance with another aspect of the present invention, in an inspection area generated every time the gantry rotates through 360 degrees, each row of detectors is directed to inspect a sector section of 360/N degrees of the inspection area, and every time the gantry rotates through 360/N degrees, an object under inspection is moved by means of the transfer device by a length equal to a distance between centers of the adjacent rows of detectors so that the respective sector sections of 360/N degrees are inspected by the N rows of detectors in a sequence from a first row of detectors of the N rows of detectors on an upstream side in a movement direction of the transfer device to a last row of detectors of the N rows of detectors.

In accordance with a further aspect of the present invention, the inspection system further comprises a scanning imaging device for obtaining a two-dimension image of an object under inspection. The CT apparatus and the scanning imaging device can operate simultaneously so that a three-dimension image and a two-dimension image of an object under inspection can simultaneously be obtained by the CT apparatus and the scanning imaging device, respectively.

In an embodiment of the present invention, the CT apparatus and the scanning imaging device can operate simultaneously when an object under inspection moves at a speed of 0.18-0.25 m/s.

In accordance with an aspect of the present invention, there is provided an inspection method comprising the steps of: transferring an object under inspection, inspecting the object by means of a CT apparatus. The CT apparatus includes a gantry, a radiation source connected with the gantry, and a detection device connected with the gantry opposite to the radiation source. The detection device comprises N rows of detectors arranged at predetermined intervals, where N is an integer greater than 1.

In accordance with another aspect of the present invention, every time the gantry rotates through 360/N degrees, an object under inspection is moved by means of the transfer device by a length equal to a distance between centers of the adjacent rows of detectors so that respective sector sections of 360/N degrees are inspected by the N rows of detectors in a sequence from a first row of detectors of the N rows of detectors on an upstream side in a movement direction of the transfer device to a last row of detectors of the N rows of detectors.

In accordance with a further aspect of the present invention, the inspection method further comprises inspecting an object under inspection by means of a scanning imaging device for obtaining a two-dimension image of an object under inspection. The CT apparatus and the scanning imaging device can operate simultaneously so that a three-dimension image and a two-dimension image of an object under inspection can simultaneously be obtained by the CT apparatus and the scanning imaging device, respectively.

In an embodiment of the present invention, the CT apparatus and the scanning imaging device can operate simultaneously when an object under inspection moves at a speed of 0.18-0.25 m/s.

In accordance with an aspect of the present invention, there is provided a CT apparatus comprising a gantry, a radiation source connected with the gantry, and a detection device connected with the gantry opposite the radiation source. The detection device comprises N rows of detectors arranged at predetermined intervals, where N is an integer greater than 1.

In accordance with an aspect of the present invention, there is provided a detection device for a CT apparatus, comprising: N rows of detectors with a predetermined interval between two adjacent rows of detectors, where N is an integer greater than 1.

In one embodiment of the present invention, the predetermined interval may be at least about 5 mm and at most about 80 mm, and in another embodiment, the predetermined interval may be at least about 30 mm and at most about 50 mm.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 is a schematic view of an inspection system according to an embodiment of the present invention.

FIG. 2 is a schematic view of CT apparatus according to an embodiment of the present invention.

FIG. 3 is a schematic view of a detection device according to an embodiment of the present invention.

FIG. 4 is a top view showing an arrangement of detectors of a detection device according to an embodiment of the present invention.

FIG. 5 is a schematic view showing a structure of a scintillation detector according to an embodiment of the present invention.

FIG. 6 is a schematic top view of the scintillation detector shown in FIG. 5.

FIG. 7 is a perspective view of the scintillation detector shown in FIG. 5.

FIG. 8 is a schematic top view of a detection device with a single row of detectors.

FIG. 9 is a schematic top view of a detection device having a plurality of rows of detectors with a wide interval between the adjacent rows of detectors.

### DETAILED DESCRIPTION OF THE PREFERRED EMBODIMENTS

Reference will now be made in detail to the exemplary embodiments of the present invention, an example of which is illustrated in the accompanying drawings, wherein like reference numerals refer to like elements throughout. The exemplary embodiments are described below to explain the present invention by referring to the accompanying drawings.

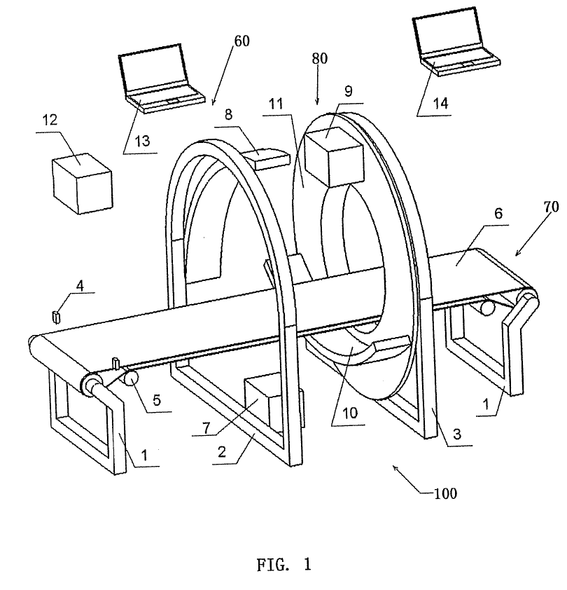

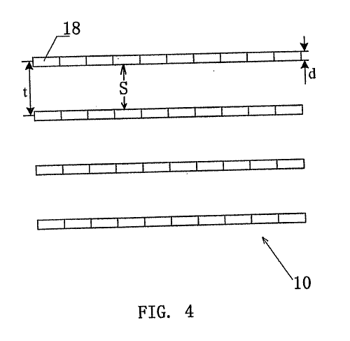

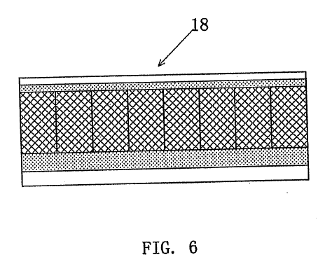

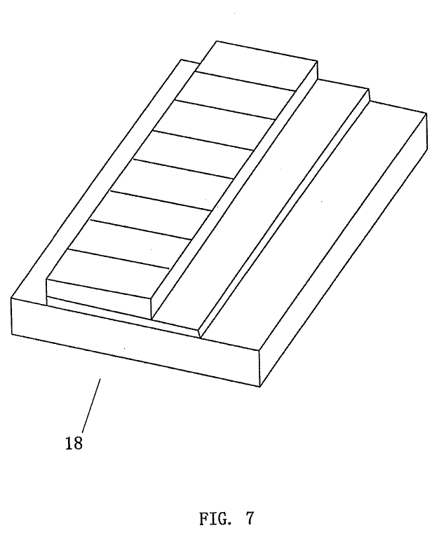

Referring to FIGS. 1-8, an inspection system 100 according to an embodiment of the present invention comprises a CT apparatus 80. The CT apparatus 80 includes a gantry 11, a radiation source 9 connected with the gantry 11, a detection device 10 connected with the gantry 11 substantially opposite to the radiation source 9, and a transfer device 6 for transferring an object under inspection. The detection device 10 comprises N rows of detectors 18 arranged at predetermined intervals, where N is an integer greater than 1. In FIG. 4, four rows of detectors 18 are shown.

In an embodiment according to the present invention, the inspection system 100 further comprises a scanning imaging device 60 for obtaining a two-dimension image of an object under inspection. The CT apparatus 80 and the scanning imaging device 60 can operate simultaneously so that a three-dimension image and a two-dimension image of an object under inspection can simultaneously be obtained by the CT apparatus 80 and the scanning imaging device 60, respectively.

In an embodiment shown in FIG. 1, the inspection system 100 according to the present invention further comprises a scanning imaging device 60 for obtaining a two-dimension transmission image of an object under inspection and a CT apparatus 80. The scanning imaging device 60 may be any appropriate imaging apparatuses known to the art such as single-energy and dual-energy scanning imaging device. The inspection system 100 can inspect contraband items such as explosives and drugs. The CT apparatus 80 can accurately obtain information such as three-dimensional shape and size, effective atomic member (Z) and density (D) of an object under inspection. Contraband items such as explosives and drugs can be judged on the basis of a plot of effective atomic member (Z) versus density (D) of the items. In addition, the CT apparatus employs multiple rows of detectors to improve a scanning speed and a through-rate of items under inspection to a great extent.

The inspection system 100 according to an embodiment of the present invention may further comprise a belt conveyor 70 composed of a support 1, a belt 6, and a belt position coder 5.

In an embodiment according to the present invention, the scanning imaging device 60 comprises a support 2, a radiation source 7 connected with the support 2, a detection and data acquisition unit 8 connected to the support 2 opposite to the radiation source 7.

In an embodiment according to the present application, the CT apparatus 80 comprises a support 3, a gantry 11 rotatably coupled with the support 3, a radiation source 9 connected with the gantry 11, and a detection and data acquisition unit 10 (that is, an example of the detection device 10) connected to the gantry 11 opposite to the radiation source 9.

In addition, the inspection system 100 according to an embodiment of the present invention may further comprise an item positioning device 4 for determining a position of an item, a control module 12 for controlling the inspection system 100, a computer data processor 13 for processing data obtained by the scanning device 60, and a computer data processor 14 for processing data obtained by the CT apparatus 80.

The item positioning device 4 may comprise a photoelectric sensor or other devices for judging a starting point and an end point of an item under inspection. The item positioning device 4 can cooperate with the belt position coder 5 to determine a position of an item in a tunnel (not shown).

The detection and data acquisition units 8 and 10 are integral module. A data acquisition section of each of the detection and data acquisition units 8 and 10 comprises a signal amplifier circuit, an A/D (Analog-to-Digital) conversion circuit, and a data transmission circuit.

In an embodiment according to the present application, the radiation source 7 is disposed on a side of the tunnel, while the detection and data acquisition unit 8 is disposed on another side of the tunnel just opposite to a beam of radiation emitted from the radiation source 7. Both the radiation source 9 and the detection and data acquisition unit 10 are fixed on the gantry 11 in such a manner that the detection and data acquisition unit 10 is oriented just opposite to a beam of radiation emitted from the radiation source 9.

The control module 12 communicates with the item positioning device 4, the belt position coder 5, the belt conveyor 70, the radiation source 7, detection and data acquisition unit 8, the radiation source 9, detection and data acquisition unit 10, the gantry 11, the computer data processor 13, and the computer data processor 14 and synchronously controls their operation states.

A data output cable of the detection and data acquisition unit 8 is connected to the computer data processor 13, and a data output cable of the detection and data acquisition unit 10 is connected to the computer data processor 14.

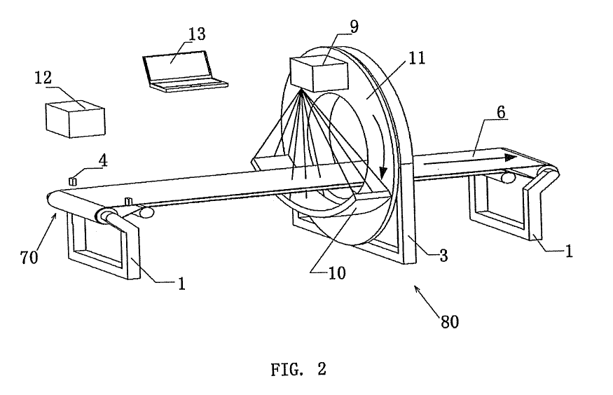

The inspection system 100 according to an embodiment of the present invention can comprise only a CT apparatus 80 as shown in FIG. 2.

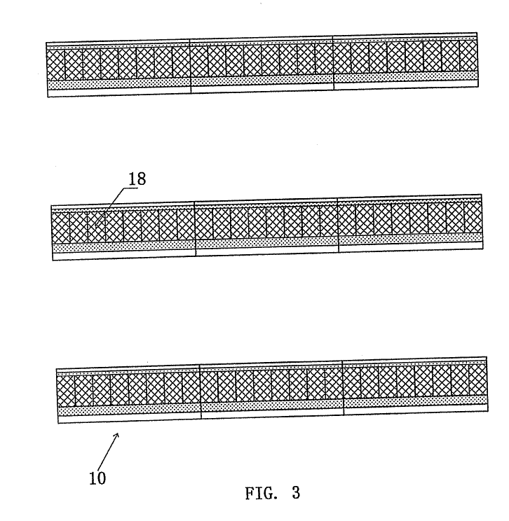

Referring to FIGS. 3-4, the detection device 10 according to an embodiment of the present invention comprises a plurality of rows of detectors 18 arranged at predetermined intervals. The plurality of rows of detectors 18 can be arrayed in a general arc in across-section. The plurality of rows of detectors can be arrayed in any manners known to the art as long as a plurality of rows of detectors 18 are arrayed at predetermined intervals.

Referring to FIG. 4, t represents a center distance between two adjacent rows of detectors 18, for example in a moving direction of the belt 6 shown in FIG. 1, and d represents a width of each of the detectors 18, for example in the moving direction of the belt 6 shown in FIG. 1. The interval S is equal to a difference between the center distance t and the width d. That is, S=t−d.

In some embodiments, the t is set to be greatly more than d, i.e. t>>d, where t is the distance between the two adjacent detectors 18 of the plurality of two detectors 18 of the detection device 10, and d is the width of the detectors 18. Therefore, an area of a scintillation crystal of scintillation detectors of the detecting device 10 decreases, thereby reducing the cost of the detection device. The detection device 10 multiples in detection rate compared with a detection device with a single row of detectors. It is apparent that a spatial resolution is reduced when t>>d. However, the reduction of spatial resolution is allowable under relevant laws since a low spatial resolution is required when detecting some items such as explosives. For example, explosives having a size smaller than a dimension do not constitute a security threat.

In an example of the present invention, the predetermined interval S may be 5 to 80 mm. In another example of the present invention, the predetermined interval S may be 10 to 70 mm. In a further example of the present invention, the predetermined interval S may be 20 to 60 mm. In a still further example of the present invention, the predetermined interval S may be 30 to 50 mm. In another example of the present invention, the predetermined interval S may be 35 to 45 mm. In a further example of the present invention, the predetermined interval S may be 36 to 40 mm or about 38 mm.

The predetermined interval varies depending upon requirements for inspection. For example, when explosives are to be detected, if the width d of the detectors is 2 mm and the interval S is about 38 mm or about 40 mm, the explosives do not constitute a threat and are allowable under relevant laws. The interval S can be determined for inspecting knifes and guns in accordance with practical situations and laws.

Generally, a width of detectors is 1-10 mm. The arrangement of the plurality rows of detectors may be defined by the center distance instead of the interval. In an example, the center distance may be 15-65 mm. In another example, the center distance may be 25-55 mm.

The above arrangement of a detector according to the present invention is applicable to detectors such as a scintillation detector.

The structure of a detector according to the present invention will be illustrated by taking a scintillation detector as an example.

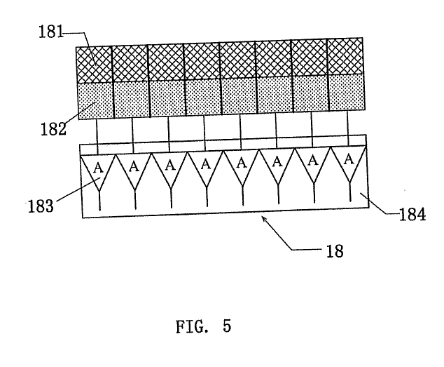

As shown in FIGS. 5-8, the scintillation detector comprises a scintillation crystal 181, a photodiode 182, and a preamplifier 183 disposed on a circuit board 184. The scintillation crystal 181 converts X-ray radiation to light. The light is converted into an electrical signal through the photodiode 182. The electrical signal is amplified through the preamplifier 183 and then transmitted to the following circuit to be proceeded.

Generally, the scintillation crystal has a small size and a big detector is achieved by splicing small modules in consideration of process and cost, thereby reducing cost and becoming convenient in maintenance.

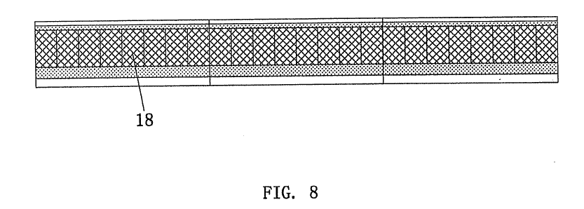

FIGS. 5-7 show a detector module 18. As shown in FIG. 8, a plurality of the detector modules 18 is spliced to constitute a signal row of detector 18. The signal row of detector 18 may be arranged in a straight line or an arc.

The effective width of the detection device increases by raising the interval between two adjacent rows of detectors. The interval between two adjacent rows of detectors may be set to be 80 mm in consideration of spatial resolution requirement for inspecting contraband items. In addition, when an object having a great size is inspected, the interval between two adjacent rows of detectors may be set to, be for example, greater, than 80 mm. The interval between two adjacent rows of detectors may be selected based on actual situations. The number of rows of the detectors used in the detection device can be selected based on actual rate and cost requirement.

The detection device can be used to scan such as a circular scan, a conventional helical scan and a helical scan meeting a particular condition.

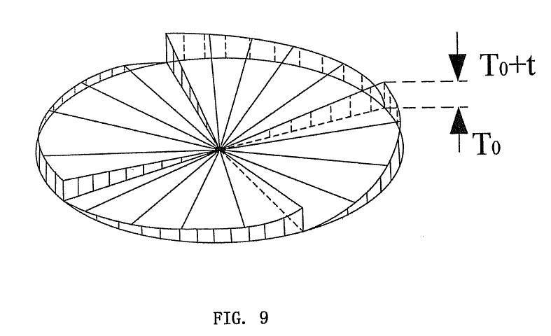

A scanning manner according to the present invention will be illuminated with reference to FIG. 9.

A scanning manner can be designed to satisfy the following equation:

$$\frac{1}{{Nr}_{0}} = \frac{t}{s} , \qquad ( 1 )$$

where t represents an interval between two adjacent rows of detectors, N represents a number of rows of detectors, r₀ represents a rotary speed of a gantry 11, and s is a speed of a belt 6.

In an inspection area generated every time the gantry rotates through 360 degrees, each row of detectors inspect a sector section of 360/N degrees of the inspection area, and every time the gantry rotates through 360/N degrees, an object under inspection is moved by means of the transfer device by a length equal to a distance between centers of the adjacent rows of detectors so that the respective sector sections of 360/N degrees are inspected by the N rows of detectors in a sequence from a first row of detectors of the N rows of detectors on an upstream side in a movement direction of the transfer device to a last row of detectors of the N rows of detectors.

If an initial position of the first row of detectors is set to be T₀, then an initial position of the second row of detector is T₀−t, an initial position of the third row of detector is T₀−2t, . . . , and an initial position of the N^{th} row of detectors is T₀−(N−1)t.

It can be found from the above equation (1) that when the gantry 11 (that is, the detection device) rotates through 360/N degrees, i.e 1/N of one rotation, the detection device relatively moves a distance of t in an axial direction. Therefore, a position of the first row of detectors becomes T₀+t, a position of the second row of detector become T₀, a position of the third row of detector become T₀−t, . . . , and an position of the N^{th} row of detectors is T₀−(N)t. In other words, the n+1^{th} row of detectors are positioned at a place at which the n^{th} row of detectors are located before the gantry 11 (that is, the detection device) rotates through 360/N degrees, and the n+1^{th} row of detectors rotate through 360/N degrees. Therefore, when the gantry rotates through 360 degrees, the N rows of detectors just cover 360 degrees from T₀ to T₀+t.

The following specific scanning steps will be illustrated.

1. A rotary speed of the gantry is set to be r₀(r/s) and a speed of the belt 6 is set to be s(m/s) so that the rotary speed of the gantry and the speed of the belt satisfy the following equation:

$$\frac{s}{{r}_{0}} = Nt , \qquad ( 2 )$$

where t represents a distance between two adjacent rows of detectors and N represents the number of rows of detectors.

2. Control motors are actuated to rotate the gantry and the belt at uniform speeds as set above, respectively.

3. When the gantry rotates to an angular position which is set to be 0 degree, a radiation source is controlled to emit X-ray and the detection device is activated to collect data. For the purpose of clarity illustration, supposing that the first rows of detectors are set as a reference, but the present invention is not limited thereto. A position of the first row of detectors relative to the belt is T₀, a position of the second row of detectors relative to the belt is T₀−t, . . . , and a position of the N^{th} row of detectors relative to the belt is T₀−(N−1)t.

4. The gantry rotates from 0 degree to 360/N degrees so that the detection device continuously collects data over the range of 0 to 360/N degrees. Since the rotary speed of the gantry and the speed of the belt satisfy the equation (2), the belt moves a distance of t. The first row of detectors collect data over an angular range of 0 to 360/N degrees in an area of T₀ to T₀+t in a direction in which the belt moves. When the gantry rotates to 360/N degrees, the position of the first row of detectors relative to the belt is T₀+t, the position of the second row of detectors relative to the belt is T₀, . . . , and the position of the N^{th} row of detectors relative to the belt is T₀−(N−2)t.

5. The gantry rotates from 360/N degrees to 2×360/N degrees during which the detection device continuously collects data over the range of 360/N to 2×360/N degrees. It can be known from the above step 4 that the second row of detectors collect data over an angular range of 360/N to 2×360/N degrees in the area of T₀ to T₀+t in the direction in which the belt moves. When the gantry rotates to 2×360/N degrees, the position of the first row of detectors relative to the belt is T₀+2t, the position of the second row of detectors relative to the belt is T₀+t, . . . , and the position of the N^{th} row of detectors relative to the belt is T₀−(N−3)t.

6. Similar to the above steps 4-5, the gantry continuously rotates. After the N+1^{th} row of detectors have collected data over an angular range of

$$\frac{360}{N} \times ( N - 2 ) to \frac{360}{N} \times ( N - 1 )$$

degrees in the area of T₀ to T₀+t, the position of the N^{th} row of detectors relative to the belt is T₀.

7. After the N^{th} row of detectors have collected data over an angular range of

$$\frac{360}{N} \times ( N - 1 ) to \frac{360}{N} \times N$$

degrees in the area of T₀ to T₀+t, the detection device completes a cycle of the data collection.

8. It can be known from the above steps 4-7 that the N rows of detectors are used to collect data over an angular range of 0 to 360 degrees in the area of T₀ to T₀+t. FIG. 9 shows an example in which N=4. A computed tomography image of the area of T₀ to T₀+t can be obtained from the data by computed tomography reconstruction.

9. Since the gantry and the belt operate continuously, the above steps 4-7 are continually conducted to obtain computed tomography images at various positions of the object under inspection.

A scanning manner according to an embodiment of the present invention will be illustrated by taking a detection device with four rows of detectors with reference to FIG. 9.

The four rows of detectors scan over an angular range of 360/4=90 degrees of 360 degrees, respectively. The interval t between two adjacent rows of detector is 40 mm.

The rotation speed of the gantry r₀ is 1.5 r/s. A scan rate is given by

s=Nr₀t.

s=4×1.5×0.04=0.24 m/s.

Data obtained under the above conditions can be used to reconstruct an image of an object under inspection by a cone-beam reconstruction algorithm taking a divergence of a cone beam into consideration.

When a distance between a detection device and a radiation source is 1000 mm, the maximal divergence is

$$γ = arc tan ( \frac{40}{1000} ) = 2.29 ° , ,$$

which is less than an empirical limit divergence of 5 degrees for a circular-scan cone-beam reconstruction. Therefore, no serious reconstruction pseudo image will be generated.

According to a normal helical scan reconstruction method, a speed of the belt (s) is given by

$$s = {pr}_{0} \frac{q}{λ} = 2 * 1.5 * \frac{120 mm}{2} = 0.18 ( m \text{/} s ) ,$$

where λ represents magnification ratio (λ>1) and is set to be 2;
q represents an effective width of a detection device and is set to be 120 mm, an equivalent width of the effective width is 60 mm at the center of the gantry;
r₀ represents the rotation speed of the gantry and is set to be 1.5 r/s;
p represents a pitch and is set to be 2 which is a maximal pitch for known image reconstruction algorithms.

It can be known from the above contents that the scanning method according to the present invention can effectively improve the scanning rate.

The CT apparatus and the scanning imaging device for obtain two-dimensional image of an object under inspection can operate simultaneously when an object under inspection moves at a speed of 0.18-0.25 m/s.

In the CT apparatus as shown in FIGS. 1-2, if the rotary speed of the gantry is 1.5 r/s, a distance between a focus of beam of the radiation source 9 and the center of the gantry is 500 mm, a distance between the focus of beam of the radiation source 9 and the detection device is 1000 mm, then the magnification ratio λ=1000/500=2.

If a detection device with four rows of detectors is employed, the width d of the crystal of the detectors is 2 mm, and the distance t between the centers of the adjacent two rows of detectors is 40 mm, then the entire width q of the detection device is 120 mm. If the reconstruction is performed when the pitch p=2, the speed of the belt is given by:

s=p*r₀*(q/λ)=2*1.5*(0.120/2)=0.18 m/s

The pitch p is an important parameter for a helical orbit that is produced when a helical scan is performed. The pitch has been defined in many ways in prior art. In the present invention, the pitch p is defined as a radio of a distance between adjacent two turns of the helical orbit to the effective width of the detecting device

In most of commercial inspection systems, a CT apparatus and a scanning imaging device for obtaining a two-dimensional image of an object under inspection can not simultaneously operate due to large difference in scanning imaging rate. Generally, when the scanning imaging device has detected a suspicious object, the CT apparatus is used to further scan the object, which will increase a rate of failure of detection of the system. However, when the CT apparatus according to the present invention is employed, the CT apparatus can perform scanning imaging at a high rate to enable the CT apparatus and the scanning imaging device for obtaining a two-dimensional image of an object under inspection to simultaneously operate, thereby compensating each other's insufficiency.

If the inspection system according to the present invention has a resolution of 20 mm in a Z direction (horizontal direction) and a resolution of more than 10 mm in an XY direction (a vertical plane), a minimal volume of an object that the system can detect is about 10 cm³. Common explosives have a density of 1.5-1.9 g/cm³ so that the system can detect a minimal explosive of 20 g. The system can detect a minimal explosive of 50 g in consideration of influence of factors such as system noise.

An inspection method according to an embodiment of the present invention will be illustrated with reference to FIGS. 1, 2, 4, and 9.

An inspection method according to an embodiment of the present invention comprises the steps of transferring an object under inspection, inspecting the object by means of a CT apparatus. The CT apparatus includes a gantry, a radiation source connected with the gantry, and a detection device connected with the gantry opposite to the radiation source. The detection device comprises N rows of detectors arranged at predetermined intervals, where N is an integer greater than 1.

In one embodiment, every time the gantry rotates through 360/N degrees, an object under inspection is moved by means of the transfer device by a length equal to a distance between centers of the adjacent rows of detectors so that respective sector sections of 360/N degrees are inspected by the N rows of detectors in a sequence from a first row of detectors of the N rows of detectors on an upstream side in a movement direction of the transfer device to a last row of detectors of the N rows of detectors.

The inspection method may further comprise inspecting an object under inspection by means of a scanning imaging device for obtaining a two-dimension image of an object under inspection. The CT apparatus and the scanning imaging device can operate simultaneously so that a three-dimension image and a two-dimension image of an object under inspection can simultaneously be obtained by the CT apparatus and the scanning imaging device, respectively. In one embodiment, the CT apparatus and the scanning imaging device can operate simultaneously when an object under inspection moves at a speed of 0.18-0.25 m/s.

The operation of an inspection system according to an embodiment will be illustrated with reference to FIGS. 1-2.

1. The item positioning device 4, the belt position coder 5, the belt conveyor 70, the radiation source 7, the detection and data acquisition unit 8, the radiation source 9, the detection and data acquisition unit 10 (an example of the detection device 10), the gantry 11, the computer data processor 13, and the computer data processor 14 all of which are controlled by the control module 12 are energized. The belt moves at a high speed and the gantry 11 begins to rotate at a predetermined rotation speed under the control of the control module 12, and then a baggage is placed on the belt.

2. When the baggage is moved to the item positioning device 4, the item positioning device 4 determines a starting point of the baggage. The control module 12 tracks a position of the baggage in real time based on the starting point and counting conducted by the belt position coder 5. When the baggage leaves the item positioning device 4, the item positioning device 4 determines an end point of the baggage. The control module 12 calculates a length of the baggage according to the starting point and the end point of the baggage.

3. When the baggage approaches a plane in which the radiation source 7 and the detection and data acquisition unit 8 are located, the radiation source 7 begins to emit a beam of radiation. The beam of radiation emitted by the radiation source 7 penetrates the baggage under inspection and is received by the detection and data acquisition unit 8 just opposite to the beam of radiation to form projection data. The control module 12 controls the detection and data acquisition unit 8 to perform measurement at a sampling rate. The measured projection data are transmitted to the computer data processor 13. When the end point of the baggage leaves the plane in which the radiation source 7 and the detection and data acquisition unit 8 are located, the radiation source 7 stops emitting a beam of radiation.

4. The computer data processor 13 corrects the projection data, and reconstructs two-dimensional images of the baggage under inspection by means of the corrected projection data.

5. When the baggage approaches a plane in which the gantry 11 is located, the radiation source 9 begins to emit a beam of radiation. The beam of radiation emitted by the radiation source 9 penetrates the baggage under inspection and is received by the detection and data acquisition unit (an example of the detection device 10) 10 just opposite to the beam of radiation to form projection data. The control module 12 controls the gantry 11 to rotate at a predetermined speed, and at the same time control the detection and data acquisition unit 10 to perform measurement at a sampling rate. The measured projection data are transmitted to the computer data processor 14. When the end point of the baggage leaves the plane in which the gantry 11 is located, the radiation source 9 stops emitting a beam of radiation. In an example, when the baggage approaches a plane in which the gantry 11 is located, the belt decelerates to move in a lowered speed, and the belt accelerates to move in an increased speed after the radiation source 9 stops emitting a beam of radiation.

6. When it can not be judged whether or not the baggage contains an explosive or a drug based on the two-dimensional image, the computer data processor 14 corrects the projection data, and obtains information on an effective atomic member and a density of an item contained in the baggage by reconstruction. Whether or not the baggage contains an explosive or a drug is finally judged by comparing the obtained information with data of contraband items stored in a data bank and by referring a shape and a size of a suspicious object. Inspected information of contents of the baggage under inspection is visually displayed from the two-dimensional projection images and a suspicious object will be marked on the projection two-dimensional images if there is the suspicious object.

With the detection device according to the present invention, an inspector can be provided with not only familiar two-dimensional images, but also accurate three-dimensional images reconstructed with a CT apparatus, thereby providing the inspector with a comprehensive accurate evidences for judging whether or not explosives and drugs are concealed in a baggage.

What is claimed is:

1. An inspection system, comprising:
a CT apparatus, the CT apparatus including a gantry, a radiation source connected with the gantry, a detection device connected with the gantry substantially opposite to the radiation source, and
a transfer device for transferring an object under inspection,
wherein the detection device comprises N rows of detectors with a predetermined interval between two adjacent rows of detectors, where N is an integer greater than 1.

2. The inspection system according to claim 1, wherein
the predetermined interval is at least about 5 mm and at most about 80 mm.

3. The inspection system according to claim 1, wherein
the predetermined interval is at least about 30 mm and at most about 50 mm.

4. The inspection system according to claim 1, wherein
in an inspection area generated every time the gantry rotates through 360 degrees, each row of detectors inspect a sector section of 360/N degrees of the inspection area, and every time the gantry rotates through 360/N degrees, an object under inspection is moved by means of the transfer device by a length equal to a distance between centers of the adjacent rows of detectors.

5. The inspection system according to claim 1, further comprising a scanning imaging device for obtaining a two-dimension image of an object under inspection,
wherein the CT apparatus and the scanning imaging device can operate simultaneously so that a three-dimension image and a two-dimension image of an object under inspection can simultaneously be obtained by the CT apparatus and the scanning imaging device, respectively.

6. The inspection system according to claim 5, wherein
the CT apparatus and the scanning imaging device can operate simultaneously when an object under inspection moves at a speed of 0.18-0.25 m/s.

7. An inspection method, comprising the steps of:
transferring an object under inspection, and
inspecting the object by means of a CT apparatus, the CT apparatus including a gantry, a radiation source connected with the gantry, and a detection device connected with the gantry opposite to the radiation source,
wherein the detection device comprises N rows of detectors with a predetermined interval between two adjacent rows of detectors, where N is an integer greater than 1.

8. The inspection method according to claim 7, wherein
every time the gantry rotates through 360/N degrees, an object under inspection is moved by means of the transfer device by a length equal to a distance between centers of the adjacent rows of detectors.

9. The inspection method according to claim 7, wherein
the predetermined interval is at least about 5 mm and at most about 80 mm.

10. The inspection method according to claim 7, wherein
the predetermined interval is at least about 30 mm and at most about 50 mm.

11. The inspection method according to claim 7, further comprising inspecting an object under inspection by means of a scanning imaging device for obtaining a two-dimension image of an object under inspection,
wherein the CT apparatus and the scanning imaging device can operate simultaneously so that a three-dimension image and a two-dimension image of an object under inspection can simultaneously be obtained by the CT apparatus and the scanning imaging device, respectively.

12. The inspection method according to claim 11, wherein
the CT apparatus and the scanning imaging device can operate simultaneously when an object under inspection moves at a speed of 0.18-0.25 m/s.

13. A CT apparatus, comprising:
a gantry,
a radiation source connected with the gantry, and
a detection device connected with the gantry opposite to the radiation source,
wherein the detection device comprises N rows of detectors with a predetermined interval between two adjacent rows of detectors, where N is an integer greater than 1.

14. The CT apparatus according to claim 13, wherein
the predetermined interval is at least about 5 mm and at most about 80 mm.

15. The CT apparatus according to claim 13, wherein
the predetermined interval is at least about 30 mm and at most about 50 mm.

16. A detection device for a CT apparatus, comprising:
N rows of detectors with a predetermined interval between two adjacent rows of detectors, where N is an integer greater than 1.

17. The detection device according to claim 16, wherein
the predetermined interval is at least about 5 mm and at most about 80 mm.

18. The detection device according to claim 16, wherein
the predetermined interval is at least about 30 mm and at most about 50 mm.
